(19) United States
(12) Patent Application Publication
(10) Pub. No.: US 2010/0152636 A1
(43) Pub. Date: Jun. 17, 2010

(54) BALLISTIC LUMBAR SUPPORT BELT

(76) Inventors: Ardith D. Parks, Chickadee Court, GA (US); Chung Hong Lu-Lai, Coral Springs, FL (US)

Correspondence Address: Ostrow Kaufman & Frankl LLP;Susan Formicola, The Chrysler Building, 405 Lexington Avenue, 62nd Floor, NEW YORK, NY, 10174, US

(21) Appl. No.: 12/334,210

(22) Filed: Dec. 12, 2008

(51) Int. Cl. A61F 5/00

(52) U.S. Cl. 602/19; 602/33

(57) ABSTRACT

A ballistic lumbar support belt includes a lumbar support portion configured to cover and provide support to at least a portion of a wearer's lower back. The lumbar support portion is configured to receive one or more ballistic protection elements. A belt portion is connected to the lumbar support portion. The belt portion is configured to releasably secure the lumbar support portion around the wearer's waist.

### FIELD OF THE INVENTION

The present invention relates to ballistic resistant armor and more specifically to a ballistic lumbar support belt.

### BACKGROUND

Current military and law enforcement bullet resistant and non-bullet resistant tactical vests extend only partially over the lower back and do not provide complete lumbar coverage or lumbar support. As the wearer of a typical tactical vest moves, for example by bending or twisting, the lower back becomes exposed, placing the wearer at risk for substantial injury from gunfire, shrapnel and similar hazards. Additionally, military personnel often carry extremely heavy loads, in some instances more than 60 pounds, placing the military personnel at risk of lower back injury from bearing such a load.

The need for ballistic protection of the lower back bas been recognized by the US Army, who uses a back protector that attaches as an extension to the lower portion of the back panel of a tactical vest. Other lower back protectors have also been developed that attach to the lower back of a tactical vest with web straps and provide increased ballistic coverage for the lower back. However, none of these lower back attachments also provide lumbar support. Thus, there exists a need for a belt that provides both ballistic protection and lumbar support for military and law enforcement personnel, without greatly inhibiting mobility of the wearer.

### SUMMARY

In general, in one aspect, the invention features a ballistic lumbar support belt including a lumbar support portion configured to cover and provide support to at least a portion of a wearer's lower back. The lumbar support portion may be configured to receive one or more ballistic protection elements. A belt portion may be connected to the lumbar support portion. The belt portion may be configured to releasably secure the lumbar support portion around the wearer's waist.

One or more of the following features may be included. In some embodiments, the one or more ballistic protection elements may be one or more of a soft ballistic insert and a hard ballistic insert. The lumbar support portion may include one or more pockets configured to receive the one or more ballistic protection elements. The lumbar support portion may include an outer shell configured to receive the ballistic protection element. The outer shell may include one or more pockets configured to receive the one or more ballistic protection elements. The outer shell may include webbing disposed on an outer surface.

At least a portion of the belt portion may be disposed within the lumbar support portion. The belt portion may include a left strap having a left proximal end operably connected to the lumbar support portion and a left distal end having a first half of a removable fastener. The belt portion may further include a right strap having a right proximal end operably connected to the lumbar support portion and a right distal end having a second half of the removable fastener configured to releasably secure to the first half of the removable fastener. The first and second halves of the removable fastener may include one or more of hook and loop fasteners, zippers, snaps or buckles.

The belt portion may include a top locking tab and a bottom locking tab. The top locking tab and the bottom locking tab may be disposed on the left strap of the belt portion. The top locking tab and the bottom locking tab may be disposed on the right strap of the belt portion. The top locking tab may include a first half of a tab removable fastener and the bottom locking tab may include a second half of a tab removable fastener configured to releasably secure to the first half of the tab removable fastener.

Other features and advantages of the invention are apparent from the following description, the drawings and the claims. For example, the ballistic lumbar support belt may provide added lumbar support to a soldier carrying a heavy load while also providing ballistic protection to the lower back of the soldier. A ballistic lumbar support belt may assist in providing easier access to supplies attached to the web strapping disposed on the ballistic lumbar support belt. Additionally, the ballistic lumbar support belt may provide improved lower back protection while allowing a greater range of motion for the wearer. Further, the ballistic lumbar support belt can be configured and/or adjusted to fit all body types to provide an appropriate amount of protection for each wearer.

### BRIEF DESCRIPTION OF THE DRAWINGS

FIG. 1 is a rear view of an exemplary ballistic lumbar support belt including a ballistic plate partially disposed within a pocket.

FIG. 2 is a rear view of an exemplary ballistic lumbar support belt as worn with a tactical vest.

FIG. 3 is a top view of the ballistic lumbar support belt of FIG. 1 including a ballistic plate disposed within a pocket.

FIG. 4 is a side view of the ballistic lumbar support belt of FIG. 1 as worn by a wearer.

FIG. 5 is a front view of the ballistic lumbar support belt of FIG. 1 as worn by a wearer.

FIG. 6 is a rear view of another exemplary ballistic lumbar support.

FIG. 7 is a front view of the ballistic lumbar support of FIG. 6 as worn by a wearer.

Like reference numbers and designations in the various drawings indicate like elements.

### DETAILED DESCRIPTION

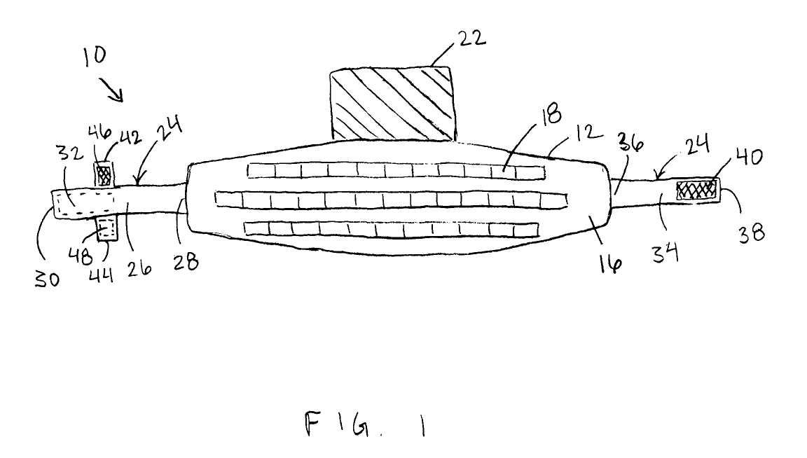

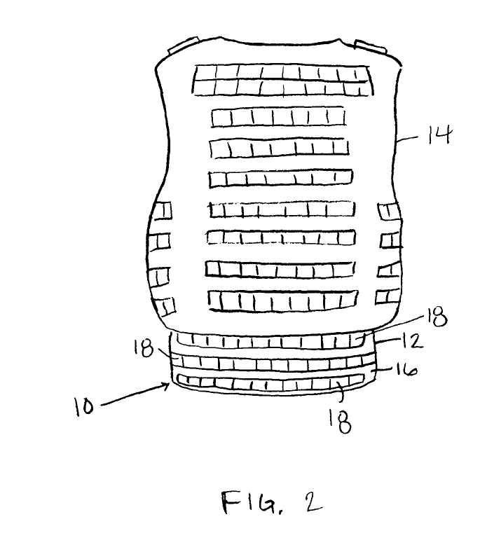

As seen in FIGS. 1, 2, and 3 a ballistic lumbar support belt 10 includes a lumbar support portion 12. The lumbar support portion 12 may be configured to cover and provide support to at least a portion of a wearer's lower back. For example, the lumbar support portion 12 may have an ergonomic shape that fits snugly around the wearer's waist, providing lumbar support similar to that provided by a weight lifting belt. The lumbar support portion 12 may be configured to fit at least partially under the lower portion of the back panel of a tactical vest 14. The lumbar support portion 12 may also be sized to wrap partially around the sides of a user, to further provide support and protection to the sides of a user.

The lumbar support portion 12 may include an outer shell 16 formed from protective fabric, such as 500d CORDURA®, or any weight of nylon protective covering such as, for example, 1000d CORDURA®, or other fabrics such as 100d microfiber and polycotton. While the outer shell 16 may be configured for comfort, it should also help protect any armor elements disposed in the lumbar support portion 12 from abrasion and deterioration. The outer shell 16 may additionally be water resistant and/or waterproof. The outer shell 16 may include webbing 18 disposed on the outside of the lumbar support portion 12, such as MOLLE straps. The webbing 18 may be configured such that equipment may be releasably secured to the lumbar support portion 12 for carrying and easy access.

The outer shell 16 of the lumbar support portion 12 may be configured to receive one or more ballistic protection elements. For example, the outer shell 16 may include one or more pockets, such as pocket 20, configured to receive a ballistic protection element 22 (shown partially disposed in the pocket 20 in FIG. 1). The ballistic protection element 22 may be a soft ballistic insert, a hard ballistic insert, or a combination of soft and hard ballistic inserts. For example, a soft ballistic insert may include any type of soft armor including, for example, soft flexible woven aramid (KEVLAR®, TWARON®, ARTEC®, etc.), woven or unidirectional laminate polyethylene's (DYNEEMA® or SPECTRA®), or any combination or hybrid thereof. For example, to provide protection from a Level IIA, II or IIIA threat (as defined in the National Institute for Justice Standard 0101.04 (“NIJ Standard”)), a soft armor insert may include an aramid fabric having a denier of 840d and an areal density of 210 oz/yd2 (1.46 psf). The aramid fabric may be made to a thickness of 0.33 inches, or to an appropriate thickness to protect against an anticipated ballistic threat. The soft ballistic insert will provide padding, as well as some of the support for the lower back. A hard ballistic insert may include one or more ceramic plates and may be used in addition, or in the alternative, to a soft ballistic insert, depending upon an anticipated threat level.

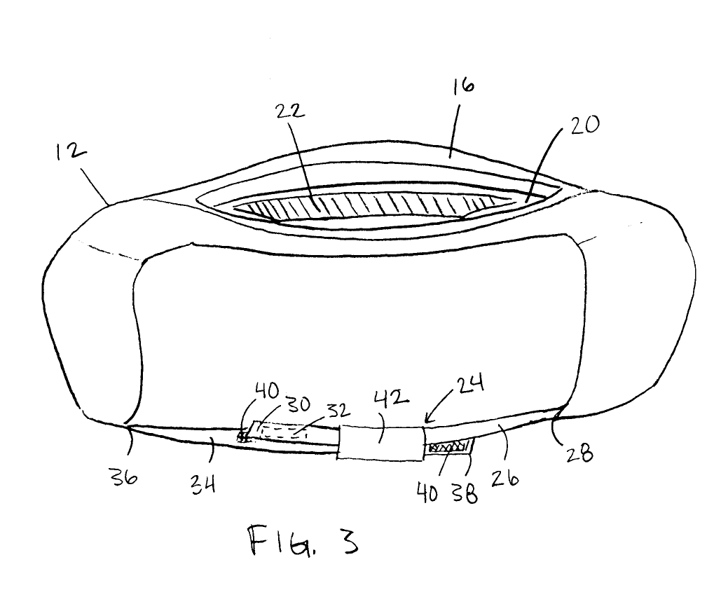

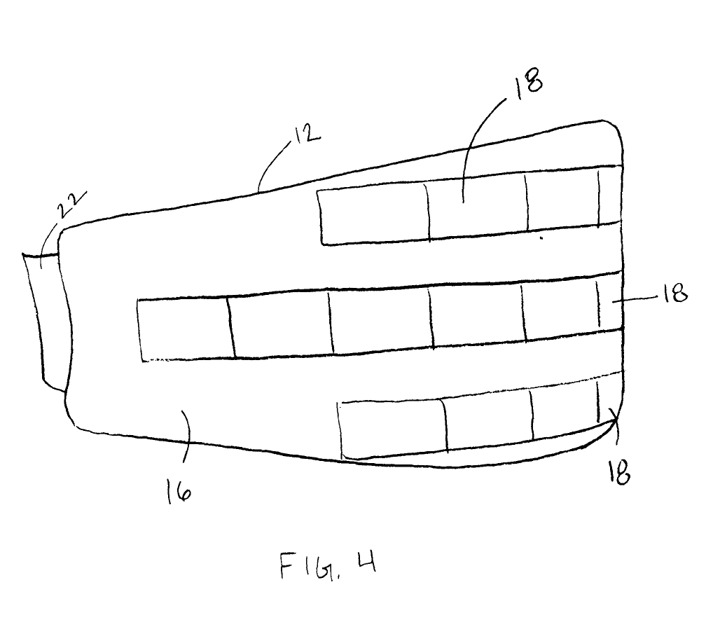

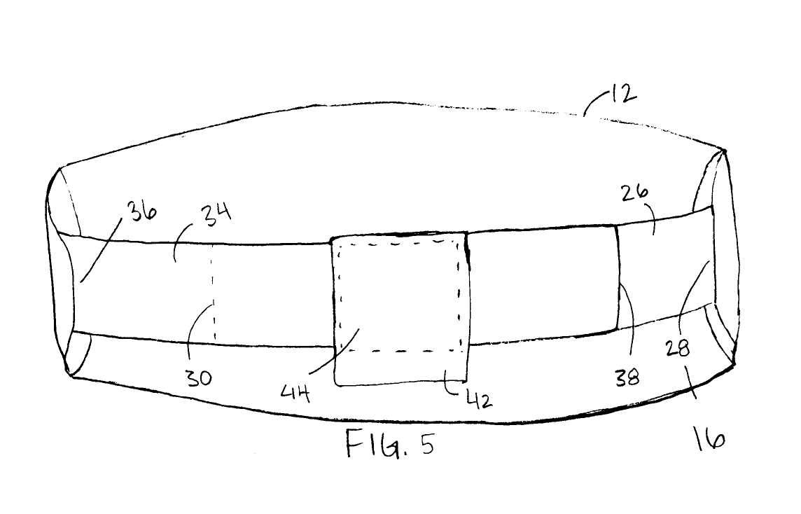

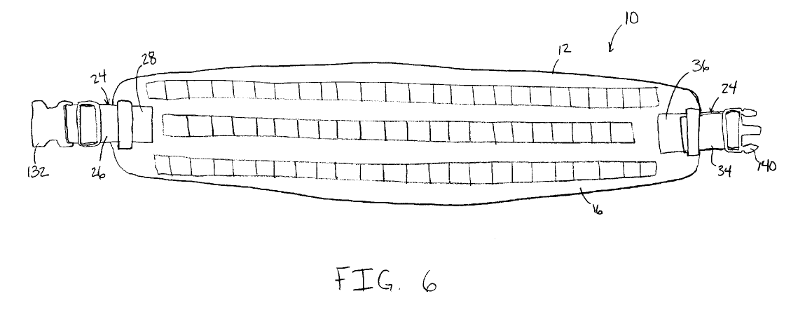

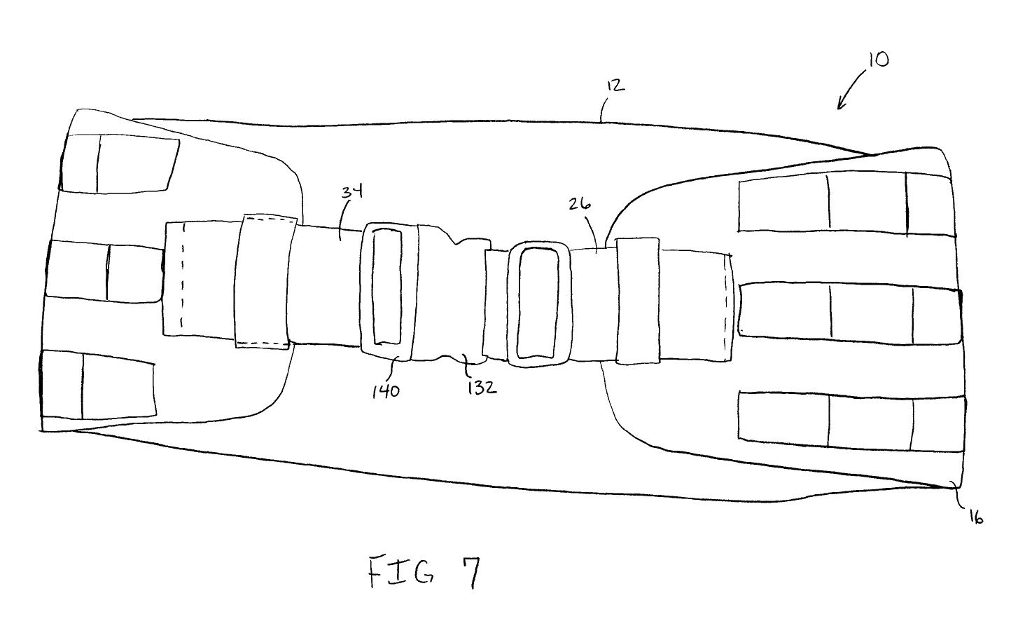

Referring also to FIG. 3, a belt portion 24 may be connected to the lumbar support portion 12. For example, the belt portion 24 may be stitched to the lumbar support portion 12. The belt portion 24 may include a left strap 26 having a left proximal end 28 operably connected to the lumbar support portion 12 and a left distal end 30 having a first half of a removable fastener 32. The belt portion 24 may further include a right strap 34 having a right proximal end 36 operably connected to the lumbar support portion 12 and a right distal end 38 having a second half of the removable fastener 40 configured to releasably secure to the first half of the removable fastener 32. The first and second halves of the removable fastener 32, 40 may include one or more of hook and loop fasteners, zippers, snaps or buckles. For example, the fastener may be the hook and loop fastener shown in FIGS. 1-5. Alternatively, in another example, the fastener may be two halves of an adjustable, quick-release, two-piece buckle 132, 140, as shown in FIGS. 6 and 7. The belt portion 24 may thereby releasably secure the lumbar support portion 12 around the wearer's waist and/or lower back. At least a portion of the belt portion 24 may be disposed within the lumbar support portion 12. For example, left proximal end 28 and right proximal end 36 may partially extend into the lumbar support portion 12 and be stitched and/or heat-sealed into attachment with the lumbar support portion 12, thereby providing a more secure attachment to the lumbar support portion 12. Alternatively, the belt portion 24 may be a single strap that runs through the interior of the lumbar support portion 12, thereby enabling slidable adjustment of the lumbar support portion 12 in relation to the belt portion 24. In an alternative embodiment, the belt portion 24 may be a single strap that is permanently attached to the lumbar support portion 12 at a proximal end, and may releasably attach to the lumbar support portion 12 at a distal end to retain the lumbar support portion 12 to the wearer.

Additionally, when a hook and loop fastener is used, the belt portion 24 may include a top locking tab 42 and a bottom locking tab 44 disposed on the left strap 26 or the right strap 34. The top and bottom locking tabs 42, 44 may be configured to fold around the left distal end 30 and the right distal end 38 when joined to prevent inadvertent opening of the closure of the belt portion 24. For example, the top locking tab 42 may include a first half of a tab removable fastener 46 and the bottom locking tab 44 may include a second half of a tab removable fastener 48 configured to releasably secure to the first half of the tab removable fastener 46.

It is to be understood that the foregoing description is intended to illustrate and not to limit the scope of the invention, which is defined by the scope of the appended claims. Other embodiments are within the scope of the following claims. For example, while the ballistic lumbar support belt has been described as including various ballistic resistant materials, it may include any other type of ballistic-resistant material or combination thereof. Further, while the ballistic resistant material was described as being of a particular denier, areal density and thickness, ballistic resistant material having any denier, areal density or thickness may be used.

What is claimed is:

1. A ballistic lumbar support belt comprising:
a lumbar support portion configured to cover and provide support to at least a portion of a lower back of a wearer, wherein the lumbar support portion comprises one or more ballistic protection elements; and
a belt portion connected to the lumbar support portion, wherein the belt portion is configured to releasably secure the lumbar support portion around the wearer.

2. The ballistic lumbar support belt of claim 1, wherein the one or more ballistic protection elements are one or more of a soft ballistic insert and a hard ballistic insert.

3. The ballistic lumbar support belt of claim 1, wherein the lumbar support portion includes one or more pockets configured to receive the one or more ballistic protection elements.

4. The ballistic lumbar support belt of claim 1, wherein the lumbar support portion includes an outer shell configured to receive the ballistic protection element.

5. The ballistic lumbar support belt of claim 4, wherein the outer shell includes one or more pockets configured to receive the one or more ballistic protection elements.

6. The ballistic lumbar support belt of claim 4, wherein the outer shell includes webbing disposed on an outer surface.

7. The ballistic lumbar support belt of claim 1, wherein at least a portion of the belt portion is disposed within the lumbar support portion.

8. The ballistic lumbar support belt of claim 1, wherein the belt portion includes:
a left strap having a left proximal end connected to the lumbar support portion and a left distal end having a first half of a removable fastener; and
a right strap having a right proximal end connected to the lumbar support portion and a right distal end having a second half of the removable fastener configured to releasably secure to the first half of the removable fastener.

9. The ballistic lumbar support belt of claim 8, wherein the first and second halves of the removable fastener include one or more of hook and loop fasteners, zippers, snaps or buckles.

10. The ballistic lumbar support belt of claim 8, wherein the belt portion includes a top locking tab and a bottom locking tab.

11. The ballistic lumbar support belt of claim 10, wherein the top locking tab and the bottom locking tab are disposed on the left strap of the belt portion proximate to the left proximal end.

12. The ballistic lumbar support belt of claim 10, wherein the top locking tab and the bottom locking tab are disposed on the right strap of the belt portion proximate to the right proximal end.

13. The ballistic lumbar support belt of claim 10, wherein the top locking tab includes a first half of a tab removable fastener and the bottom locking tab includes a second half of a tab removable fastener configured to releasably secure to the first half of the tab removable fastener.

14. The ballistic lumbar support belt of claim 1, wherein the belt portion includes a single strap configured to slidably attach to the lumbar support portion.

15. The ballistic lumbar support belt of claim 1, wherein the belt portion includes a single strap having a proximal end connected to the lumbar support portion and distal end configured to releasably connect to the lumbar support portion.

16. A ballistic lumbar support belt comprising:
a lumbar support portion configured to cover and provide support to at least a portion of a lower back of a wearer, wherein the lumbar support portion comprises an outer shell configured to form at least one pocket to removably receive one or more ballistic protection elements; and
a belt portion connected to the lumbar support portion, wherein the belt portion is configured to releasably secure the lumbar support portion around the wearer.

17. The ballistic lumbar support belt of claim 16 wherein the one or more ballistic protection elements are one or more of a soft ballistic insert and a hard ballistic insert.

18. The ballistic lumbar support belt of claim 16, wherein the belt portion includes:
a left strap having a left proximal end connected to the lumbar support portion and a left distal end having a first half of a removable fastener; and
a right strap having a right proximal end connected to the lumbar support portion and a right distal end having a second half of the removable fastener configured to releasably secure to the first half of the removable fastener.

19. The ballistic lumbar support belt of claim 18, wherein the first and second halves of the removable fastener include one or more of hook and loop fasteners, zippers, snaps or buckles.

20. The ballistic lumbar support belt of claim 16, wherein the belt portion includes a single strap configured to slidably attach to the lumbar support portion.

21. The ballistic lumbar support belt of claim 16, wherein the belt portion includes a single strap having a proximal end connected to the lumbar support portion and distal end configured to releasably connect to the lumbar support portion
